(19) United States
(12) Patent Application Publication
(10) Pub. No.: US 2012/0283802 A1
(43) Pub. Date: Nov. 8, 2012

(54) FAR-INFRARED RADIATION IN USE OF IMPROVING PATENCY OF ARTERIOVENOUS FISTULA, DECREASING FAILURE OF ARTERIOVENOUS FISTULA MATURATION, AND PREVENTING AND/OR AMELIORATING PERIPHERAL ARTERY DISEASES

(76) Inventors: Chih-Ching LIN, New Taipei City (TW); Chyi-Ran LEE, New Taipei City (TW)

(21) Appl. No.: 13/554,238

(22) Filed: Jul. 20, 2012

Related U.S. Application Data
(63) Continuation-in-part of application No. 11/896,879, filed on Sep. 6, 2007.
(60) Provisional application No. 60/885,317, filed on Jan. 17, 2007.

(51) Int. Cl. A61N 5/06

(52) U.S. Cl. 607/88

(57) ABSTRACT

The present invention provides a novel use of far-infrared radiation for improving patency of arteriovenous fistula, decreasing failure of arteriovenous fistula maturation and preventing and/or ameliorating peripheral artery diseases in a subject in need thereof. The radiation has an electromagnetic wave of about 1.5 to 100 μm wavelength, which performs on the subject skin surface for more than 10 minutes.

### CROSS REFERENCE TO RELATED APPLICATIONS

This application is a Continuation-In-Part of pending U.S. patent application Ser. No. 11/896,879, filed Sep. 6, 2007 and entitled “Method for preventing and/or ameliorating inflammation”, which claims the benefit of U.S. Provisional Application No. 60/885,317, filed Jan. 17, 2007. The disclosure of the applications is incorporated herein by reference.

### BACKGROUND OF THE INVENTION

1. Field of the Invention

The present invention relates to a novel use of far-infrared radiation for improving patency of arteriovenous fistula, decreasing failure of arteriovenous fistula maturation and preventing and/or ameliorating a peripheral artery disease in a subject in need thereof.

2. Description of the Related Art

Infrared radiation is an invisible electromagnetic wave with longer wavelengths than those of visible light, extending from the nominal red edge of the visible spectrum at 0.74 μm to 300 μm. Two common divisions of the infrared radiation have been well-known, near-infrared radiation with wavelengths of 0.8-1.5 μm and far-infrared (FIR) radiation with wavelengths of 1.5-1000 μm. The infrared radiation has been used in industrial, scientific and medical applications, such as night-vision devices, imaging camera, laser, or the like.

In medicine, the far-infrared radiation has been commonly used in holistic medicine for increasing blood flow and reducing/healing pain. The far-infrared radiation in combination of sauna has been published for treating congestive heart failure (Richard Beever, Clinical review: Far-infrared saunas for treatment of cardiovascular risk factors, summary of published evidence, Can Fam Physician 2009; 55:691-6).

Although numerous therapeutic effects of the far-infrared therapy have been advertised, scientific studies for supporting the claimed effects are little. The present inventors therefore design experiments for revealing the far-infrared therapeutic effects and find out novel therapeutic uses of the far-infrared radiation.

### BRIEF SUMMARY OF INVENTION

One embodiment of the invention provides a method for improving patency of an arteriovenous fistula in a subject in need thereof, comprising irradiating the subject skin surface above the arteriovenous fistula (AV fistula) with an electromagnetic wave from an emitter, wherein the electromagnetic wave has a wavelength of about 1.5 to 100 μm.

Another embodiment of the invention provides a method for decreasing a rate of failure of arteriovenous fistula (AV fistula) maturation in a subject in need thereof, comprising irradiating the subject skin surface above the arteriovenous fistula with an electromagnetic wave from an emitter, wherein the electromagnetic wave has a wavelength range of about 1.5 to 100 μm.

Another embodiment of the invention provides a method for preventing and/or ameliorating a peripheral artery disease (PAD) in a subject in need thereof, comprising irradiating the subject skin surface above the site that the peripheral artery disease occurs with an electromagnetic wave from an emitter, wherein the electromagnetic wave has a wavelength of about 1.5 to 100 μm.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIGS. 1A-1B show that the far-infrared radiation induces Heme Oxygenase-1 (HO-1) expression;

FIGS. 2A-2B show that the far-infrared radiation induces Nrf2 expression;

FIG. 3 show that the far-infrared radiation enhances the promoter activity of a Heme Oxygenase-1 (HO-1) gene;

FIGS. 4A-4B show that the far-infrared radiation suppresses the expression of E-selectin, VCAM-1, and ICAM-1;

FIG. 5 show that the far-infrared radiation suppresses TNF-α-induced VCAM-1;

FIG. 6A shows the relative endothelial adhesion of H-labeled U937 cells;

FIG. 6B shows the relative HO-1 expression in various groups,

FIG. 7 shows one year survival curves for unassisted patency of AV fistula for HD patients with and without far-infrared radiation treatment,

FIG. 8A shows the data of blood flow and blood pressures of AV fistula without FIR treatment, and

FIG. 8B shows the data of blood flow and blood pressures of AV fistula with the FIR treatment.

### DETAILED DESCRIPTION OF INVENTION

The following description is of the best-contemplated mode of carrying out the invention. This description is made for the purpose of illustrating the general principles of the invention and should not be taken in a limiting sense. The scope of the invention is best determined by reference to the appended claims.

In one embodiment, the invention provides a method for improving patency of an arteriovenous fistula in a subject in need thereof. In another embodiment, the invention provides a method for decreasing a rate of failure of arteriovenous fistula maturation in a subject in need thereof. The arteriovenous fistula (AV fistula) refers to a native vessel created by surgery for the connection of an artery to a vein. The patency of the AV fistula refers to a state of the AV fistula being unobstructed. The AV fistula maturation refers that AV fistula develops to a larger lumen and stronger vascular wall for hemodialysis or the like. On the contrary, the failure of AV fistula maturation is defined as an AV fistula that had been created for at least 8 weeks but not matured enough to allow successful cannulation or use during hemodialysis (Nassar G M., et al., Endovascular treatment of the “Failing to Mature” arteriovenous fistula. Clin J Am Soc Nephrol 1:275-280 (2006)). Once the AV fistula is mature, it functions as an access blood flow and is effective on hemodialysis.

It has been known that two major complications for the AV fistula, an initial failure to mature (primary nonfunction) and a later venous stenosis followed by thrombosis (Prabir R C., et al., Hemodialysis vascular access dysfunction: a cellular and molecular viewpoint, J Am Soc Nephrol 17:1112-1127, 2006). According to the invention, the far-infrared radiation (FIR) is effective for enhancing the AV fistula maturation and improving the patency of AV fistula, which becomes the powerful treatment for AV fistula. The far-infrared radiation, according to the invention, comprises an electromagnetic wave with a wavelength of about 1.5 to 1000 μm, preferably about 3 to 25 μm, irradiating on the skin surface above the AV fistula. According to the invention, the far-infrared radiation may have a wavelength peak of about 5 to 8 μm, preferably about 5 to 6μm, but not limited thereto. The distance from the emitter to the skin surface according to the invention may be about 0.1 to 60 cm, preferably about 20 to 30 cm, and the power density of the emitter is preferably lower than about 1.3 W/cm² and more preferably about 0.5 to 0.7 W/cm².

According to the invention, the FIR irradiation may be performed continuously or discontinuously. In one example, the FIR irradiation performs more than 10 min once time and preferably about 30 to 45 min once time. The frequency of the FIR irradiation may be at least once every two days and preferably one to three times a day. In one example, the FIR irradiation may last for at least one month, but it is not limited thereto. The skin surface after the FIR irradiation can reach up to 40° C. In one example, the temperature of the skin surface after the FIR irradiation is from the body temperature to 40° C. The “emitter” recited herein refers to a device for emitting the far-infrared radiation as described above without specifically limitations. A movable or portable emitter is preferable for convenience use.

Use of FIR irradiation for enhancing the AV fistula maturation and improving the patency of AV fistula can reduce operational risk and side effects. In addition, complications accompanied with the surgery and the post-operative recovery can also be prevented. A more effective use would be to combine the FIR treatment of the invention with other treatment methods in order to improve therapeutic effectiveness, such as administration of steroid anti-inflammatory drugs or non-steroidal anti-inflammatory drugs (NSAIDs).

In another embodiment, the invention provides a method for preventing and/or ameliorating a peripheral artery disease (PAD) in a subject in need thereof. In medicine, PAD refers to the obstruction of peripheral arteries, not including those in the coronary, aortic arch vasculature and brain. Often it refers to atherosclerotic blockages found in the lower extremity. PAD may also result from epithelium dysfunctions leading to stenosis, an embolism or thrombus formation. Thus, PAD may also include a subset of disease classified as microvascular diseases resulting from episodal narrowing or widening of the arteries, such as Raynaud's phenomenon or vascular spasm.

It is well-known to diagnose and prognose PAD by the ankle-brachial index (ABI) and the brachial-ankle pulse wave velocity (baPWV) (Comfort A., et al., Brachial-ankle pulse wave velocity is associated with walking distance in patients referred for peripheral arterial disease evaluation, Atherosclerosis. 2009, September; 206 (1):173-178.). The ankle-brachial index (ABI) is the ratio of systolic blood pressure (SBP) at the ankle to simultaneously measured systolic BP at the brachial artery. The brachial-ankle pulse wave velocity (baPWV) refers to the speed of pressure pulse transmission along the ankle and brachial artery wall. An ABI ratio less than 0.9 (ABI <0.9) is consistent with PAD, and baPWV more than 2,100 cm/s is relating to potential PAD. While, ABI near to 0.9 indicates that the blood flows without resistance, and PAD does not occur. It has been established that a lower ABI and a higher brachial-ankle pulse wave velocity (baPWV) are good markers to predict the risk of PAD and atherosclerosis.

According to the invention, a far-infrared radiation (FIR) is effective for preventing and/or ameliorating the peripheral artery disease (PAD) in a subject in need thereof. The far-infrared radiation, according to the invention, comprises an electromagnetic wave with a wavelength of about 1.5 to 1000 μm, preferably about 3 to 25 μm, irradiating on the skin surface above the site that the PAD occurs. According to the invention, the far-infrared radiation may have a wavelength peak of about 5 to 8 μm, preferably about 5 to 6 μm, but not limited thereto. The distance from the emitter to the skin surface according to the invention may be about 0.1 to 60 cm, preferably about 20 to 30 cm, and the power density of the emitter is preferably lower than about 1.3 W/cm² and more preferably about 0.5 to 0.7 W/cm².

According to the invention, the FIR irradiation may be performed continuously or discontinuously. In one example, the FIR irradiation performs more than 10 min once time and preferably about 30 to 45 min once time. The frequency of the FIR irradiation according to the invention may be at least once every two days and preferably one to three times a day. In one example, the FIR irradiation may last for at least one month, but it is not limited thereto. The “emitter” recited herein refers to a device for emitting the far-infrared radiation as described above without specifically limitations. A movable or portable emitter is preferable for convenience use.

Use of FIR irradiation for preventing and/or ameliorating the peripheral artery disease (PAD) can reduce operational risk and side effects. In addition, complications accompanied with the surgery and the post-operative recovery can also be prevented. A more effective use would be to combine the FIR treatment of the invention with other treatment methods in order to improve therapeutic effectiveness, such as administration of steroid anti-inflammatory drugs or non-steroidal anti-inflammatory drugs (NSAIDs).

The “subject” recited herein refers to humans and no-human mammals, such as dogs, cats, mice, rats, cows, sheep, pigs, goats or primates, and expressly includes laboratory mammals, livestock, and domestic mammals.

### EXAMPLE

### Example 1

### FIR Therapy Induces Heme Oxygenase-1 (HO-1) Expression in HUVECs

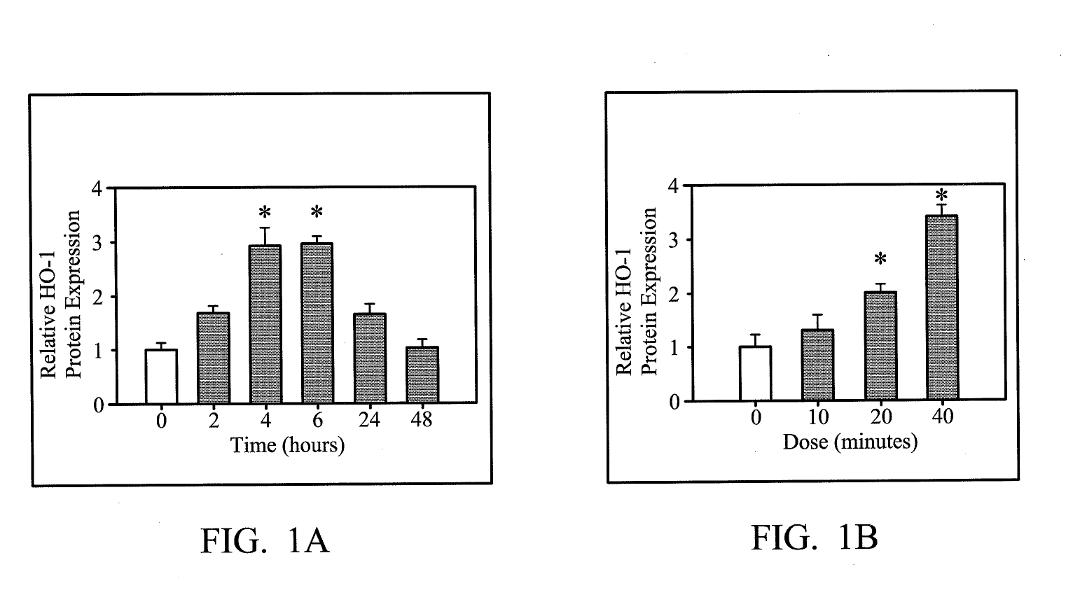

Human umbilical vein endothelial cells (HUVEC) (Walkersville, Md.) were serially cultured on gelatin-coated dishes and propagated in M199 medium supplemented with 20% bovine calf serum, 2 mM L-glutamine, 50 μg/ml endothelial cell growth factor, 90 μg/ml heparin, and 100 U/ml of penicillin and streptomycin. The cells were treated with FIR for 10 min, 20 min, 40 min using WS™ TY101 FIR emitter (WS Far Infrared Medical Technology Co., Ltd., Taipei, Taiwan). The top emitter was set at a height of 25 cm above the surface of the cells. After FIR treatment, the cells were collected at 0 h, 2 h, 4 h, 6 h, 24 h, and 48 h, and then the expression of HO-1 was analyzed by western blot and quantified by laser densitometry. The cells were lysed in a sample buffer (125 mM Tris [pH 6.8], 12.5% glycerol, 2% SDS, 50 mM sodium fluoride, and trace bromophenol blue) and proteins were separated by SDS-PAGE. Following transfer to a nitrocellulose membrane, blots were blocked with PBS and nonfat milk (5%) and then incubated with antibodies directed against HO-1 (1:500). Membranes were then washed in PBS, incubated with horseradish peroxidase-conjugated goat anti-rabbit or anti-goat antibody and developed with commercial chemoluminescence reagents (Amersham, Arlington Heights, Ill.). FIG. 1A shows that the expression of HO-1 increased significantly at 4 h after 40 minutes of FIR treatment, with the maximum effect at 6 h and this was sustained until 24 h. FIG. 1B shows that 20 and 40 minutes of FIR treatment significantly induced higher expression of HO-1 compared to 0 and 10 minutes, with the maximum increase after 40 minutes of treatment. Thus, HO-1 expression was induced in a dose-dependent manner by FIR treatment.

### Example 2

### FIR Therapy Induces Nrf2 Expression in HUVECs

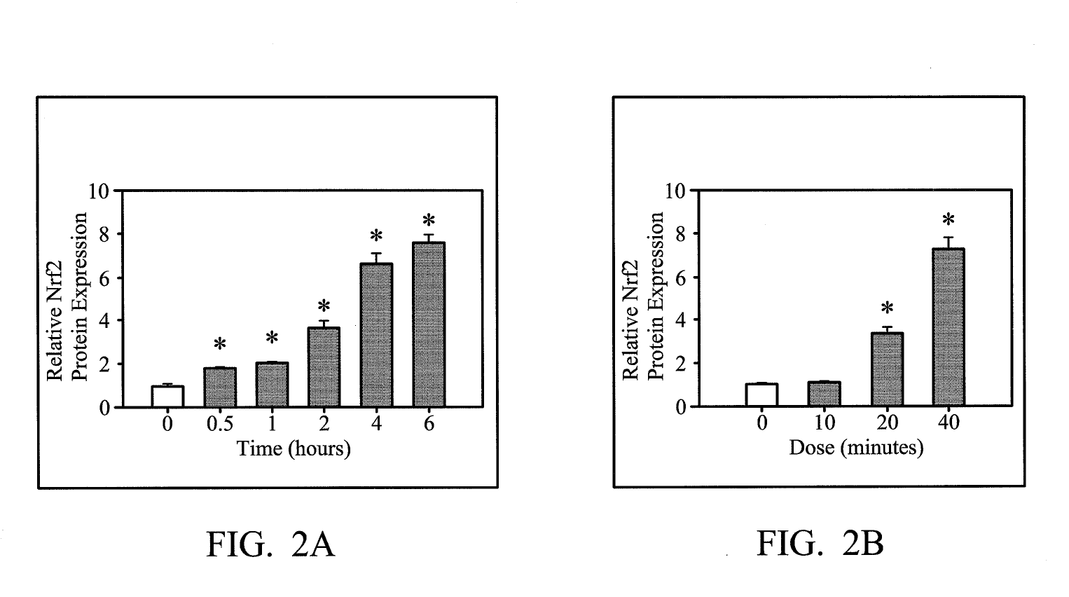

The same procedure carried out in Example 1 was repeated except that the expression of Nrf2 was detected. FIG. 2A shows that the increase of Nrf2 was time-dependent after 40 minutes of FIR therapy, with a significant increase from 0.5 h and a maximum effect at 6 h. FIG. 2B shows that 20 and 40 minutes of FIR treatment significantly induced higher expression of HO-1 than 0 and 10 minutes, with the maximum increase after 40 minutes of treatment. Thus, Nrf2 expression was induced in a dose-dependent manner by FIR treatment

### Example 3

### FIR Enhances Promoter Activity of HO-1 Gene Through Nrf2-Dependent Pathway

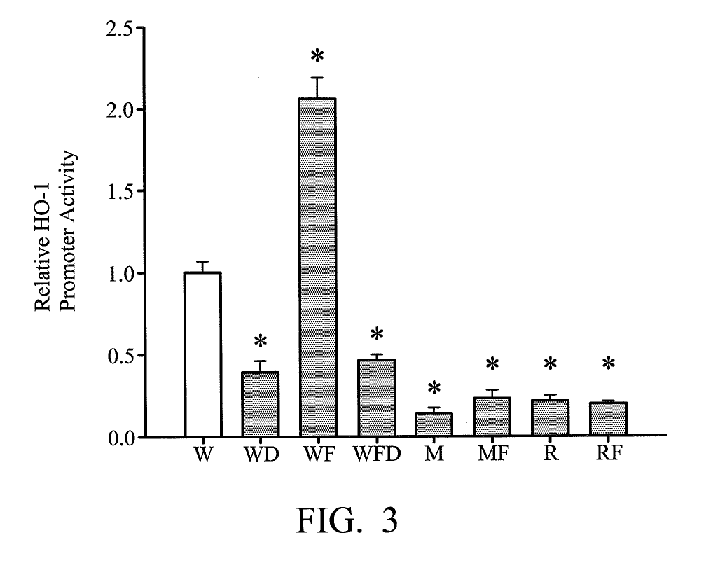

HO-1 promoter activity was determined in promoter/luciferase constructs (1 μg/ml) containing the wild type enhancer (E1) coupled to a minimum Heme Oxygenase-1 (HO-1) promoter or the mutant E1 enhancer (M739) that had its three antioxidant responsive element (ARE) core sequences mutated. These promoter constructs, pCMVβ-galactosidase (1 μg/ml), and a plasmid expressing a dominant-negative Nrf2 (dnNrf2; 1 μg/ml) that has its transactivation domain deleted were transfected into SMC using lipofectamine, and cells exposed to BHA 24 hours later. The cells were then collected, lysed, and luciferase activity measured using a dual luciferase assay system (Promega, Madison, Wis.) and a Glomax luminometer (Promega, Madison, Wis.). Firefly luciferase activity was normalized with respect to Renilla luciferase activity, and expressed as fold induction over control cells. All constructs were generously provided by Dr. Jawed Alam at the Ochsner Clinic Foundation, New Orleans, La. Referring to FIG. 3, In comparison with the wild plasmids (W) treated cells, FIR treated cells (W+F=WF) induced more than a 2-fold increase in promoter activity, which was significantly suppressed to less than 0.5-fold by adding DnNrf2 (W+F+D=WFD).

### Example 4

### FIR Treatment Suppresses TNF-α-Induced E-Selectin, VCAM-1 and ICAM-1 Expression in HUVECs

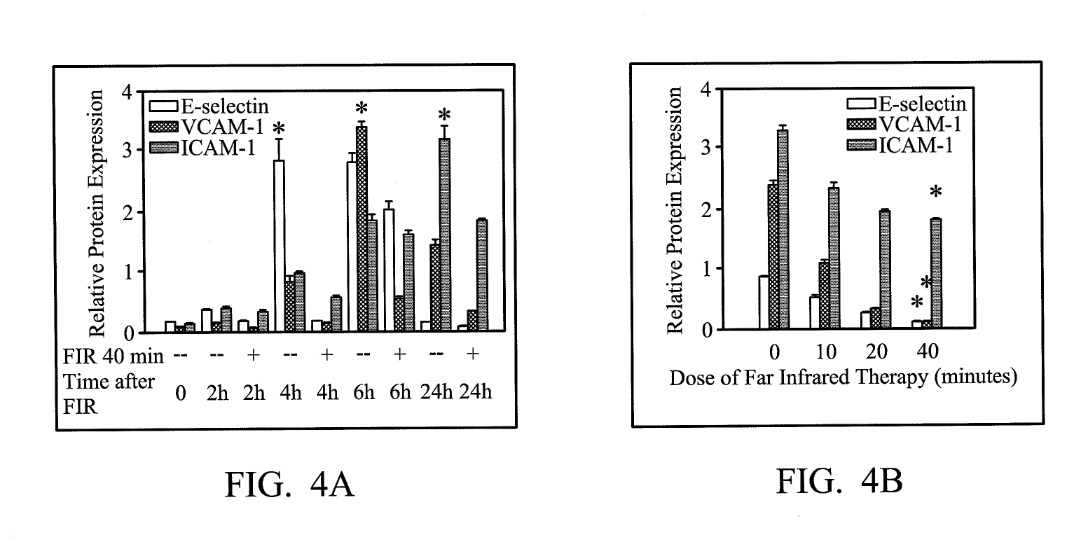

The same procedure carried out in Example 1 was repeated except that the expression of E-selectin, VCAM-1 and ICAM-1 were detected. FIG. 4A shows that the E-selectin, VCAM-1, and ICAM-1 were suppressed at hours 2, 4, 6, and 24 after 40 minutes of FIR treatment. FIG. 4B shows that the cells were collected at 4 hours for E-selectin, 6 hours for VCAM-1 and 24 hours for ICAM-1 after FIR treatment for 0, 10, 20 and 40 minutes, with the maximum inhibited response by 40 minutes of treatment for all of the three adhesion molecules. Thus, expression of E-selectin, VCAM-1 and ICAM-1 was suppressed in a dose-dependent manner by FIR treatment.

### Example 5

### FIR Suppresses TNF-α-Induced VCAM-1 Expression Through the HO-1 in HUVECs

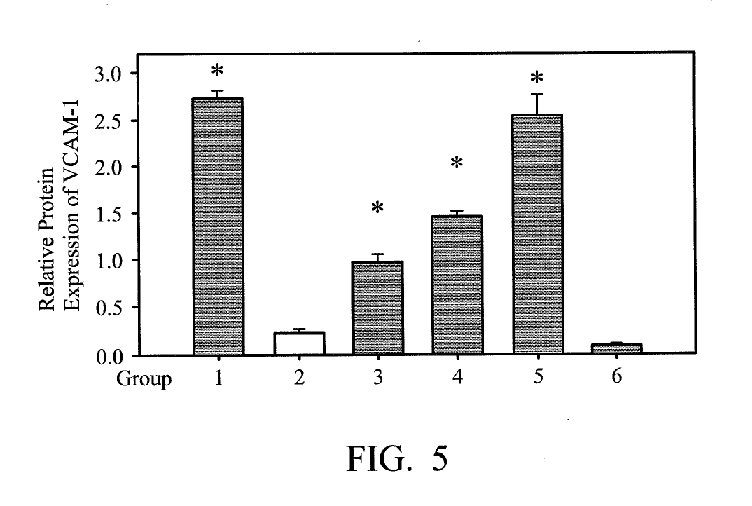

The expression of VCAM-1 was analyzed under different combinations of TNFα, FIR, HO-1 inhibitor by a western blot. HUVEC cells were classified into 6 groups according to the combination of different treatments, and the experimental condition of groups 1-6 are listed in Table 1. Referring to FIG. 5, the expression of VCAM-1 induced by TNF-α (100 ng/ml) in group 1 was significantly suppressed by adding 40 minutes of FIR in group 2. This FIR-induced inhibitory effect of VCAM-1 expression was reversed by tin protoporphyrin (SnPP, an HO-1 inhibitor) in a dose-dependent pattern, with a higher expression of VCAM-1 by 20 μM (group 4) than by 10 μM (group 3). In comparison with group 4, the expression of VCAM-1 was even higher without FIR therapy in group 5. However, a single treatment with SnPP 20 μM did not induce the expression of VCAM-1 without a pre-treatment with TNF-α (group 6).

TABLE 1

| | Group 1 | Group 2 | Group 3 | Group 4 | Group 5 | Group 6 |
|---|---|---|---|---|---|---|
| TNF-α (ng/ml) | 100 | 100 | 100 | 100 | 100 | — |
| FIR (min) | — | 40 | 40 | 40 | — | — |
| SnPP (μm) | — | — | 10 | 20 | 20 | 20 |

### Example 6

### FIR Therapy Inhibits the Adhesion of Monocytes to HUVECs Through the Stimulation of HO-1 Expression

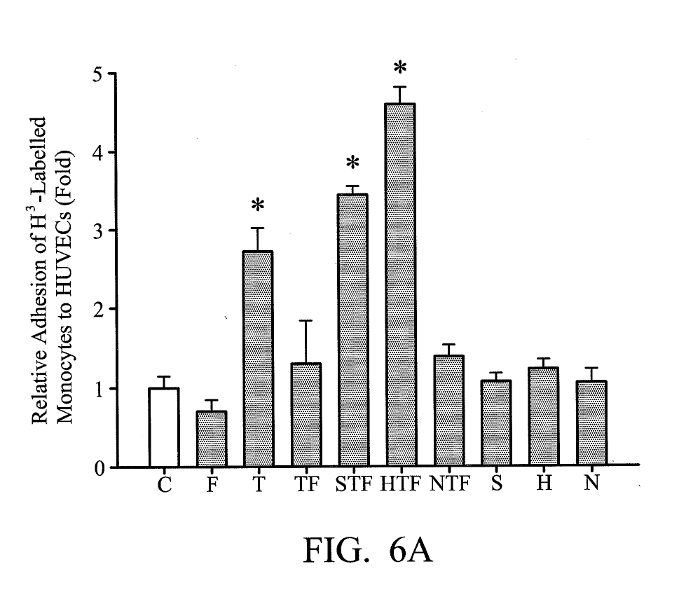

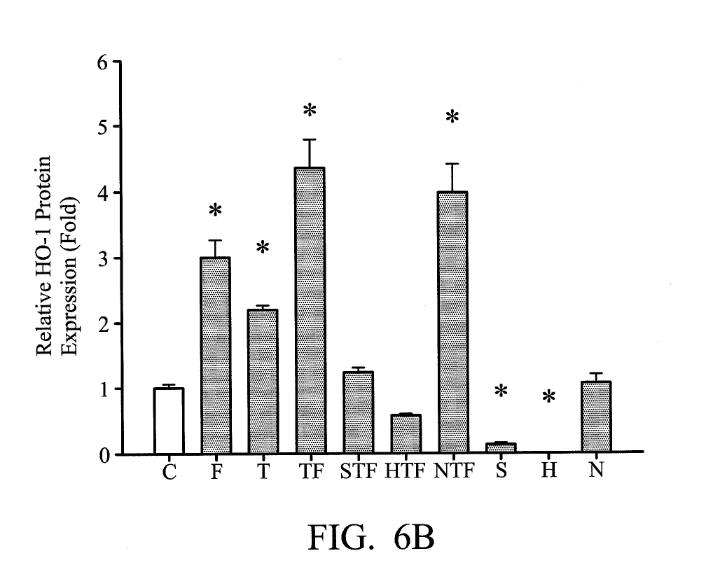

U937 cells were classified into 10 groups (F, T, TF, STF, HTF, NTF, S, H, N, or C) according to the combination of different treatment, and the experimental conditions of groups are listed in Table 2. U937 cells (1×10⁶ cells/ml) were labeled with [³H]thymidine (1 μCi/ml) for 24 hour, and then washed 3 times with a serum-free culture medium and layered onto endothelial cell monolayers that were pretreated with TNFα (100 ng/ml) for 6 hours in the presence or absence of FIR and/or SnPP. After 1 hour of incubation, nonadherent monocytes were removed by PBS washing and the radioactivity associated with adherent cells were quantified by scintillation spectrometry after lysis with 0.2% SDS/0.2 N NaOH. Additionally, the expression of HO-1 was analyzed by western blot and quantified by laser densitometry. FIG. 6A shows the relative endothelial adhesion of H-labeled U937 cells, and FIG. 6B shows the relative HO-1 expression in various groups. Referring to FIG. 6B, the FIR treatment inhibits the adhesion of H-labelled U937 cells, and the adhesion of U937 cells were increased in groups T, STF, and HTF. Referring to 6C, the HO-1 expression of F group was approximately 3-fold higher than the control group, and TF group was 4.3-fold higher than the control group. However, the HO-1 expression of the cells treated by SnPP and HO-1 SiRNA were suppressed to approximately 20% and less than 10% of that of the control cells respectively.

TABLE 2

| | Group | | | | | | | | | |
|---|---|---|---|---|---|---|---|---|---|---|
| Treatment | F | T | TF | STF | HTF | NTF | S | H | N | C |
| TNF-α | − | 100 | 100 | 100 | 100 | 100 | − | − | − | − |
| (ng/ml) | | | | | | | | | | |
| FIR (min) | 40 | − | 40 | 40 | 40 | 40 | − | − | − | − |
| SnPP (μm) | − | − | − | 20 | − | − | 20 | − | − | − |
| Non-target | − | − | − | − | − | + | − | − | + | − |
| siRNA | | | | | | | | | | |

### Example 7

### Far-Infrared Radiation (FIR) Improves Access Blood Flow and Unassisted Patency of Arteriovenous Fistula

Patient Selection

Patients included met the following criteria: (1) received 4 hours of maintenance HD therapy three times weekly for at least 6 months at Taipei Veterans General Hospital, (2) used a native AV fistula as their present vascular access for more than 6 months, without interventions within the last 3 months, and (3) creation of AV fistula by cardiovascular surgeons at Taipei Veterans General Hospital, was by standardized surgical procedures of venous end-to-arterial side anastomosis in the upper extremity. The study was based on the Helsinki Declaration [edition 6, revised 2000] and was approved by the Institutional Research Board of Taipei Veterans General Hospital. In addition, it was registered at the Cochrane Renal Group registry. After informed consent was obtained from every study subject, the patients were randomly allocated to either the group without FIR treatment or the group with FIR treatment by means of a computerized minimization algorithm to ensure balance between the two groups with respect to history of AV fistula malfunction. The subject allocation was concealed from investigators by the computerized minimization algorithm and the allocation sequence and was kept undisclosed to the investigator until the time of intervention.

Hemodialysis (HD)

All patients were dialyzed three times weekly on standard bicarbonate dialysate bath (38 mEq/L H₂CO₃, 3.0 mEq/L Ca²⁺, 2.0 mEq/L K⁺) by using the volumetric-controlled dialysis delivery system under constant dialysate flow at 500 ml/min. Patients were anticoagulated by means of systemic heparin, without change of the individual bolus or maintenance dose throughout the study.

Far-Infrared Radiation (FIR) Treatment

A WS™ TY101 FIR emitter (WS Far Infrared Medical Technology Co., Ltd., Taipei, Taiwan) was used for FIR treatment. The wavelengths generated by the FIR emitter range from about 3 μm to about 25 μm (with a peak value of about 5 μm to about 7 μm). During treatment, the top of the FIR emitter was set at a height of about 25 cm above the skin surface of the AV fistula with the treatment time set at about 40 minutes during HD three times a week.

Measurement of Hemodynamic Parameters

The access flow (Qa), cardiac output (CO) and total peripheral resistance (TPR) were measured during HD by an ultrasound dilution method using the Transonic HD02 hemodialysis monitor (Transonic Systems, Inc., Ithaca, N.Y.). The technique is widely used and validated extensively in literature. In brief, the technique uses two ultrasound sensors attached to the two HD tubing lines, one to the arterial and the other to the venous catheters, approximately 3 to 5 inches from the connection of the tubing to the dialysis needles. Initially, tubing lines are reversed, and ultrafiltration is turned off. A measured bolus of saline (10 ml) is injected into the venous catheter, resulting in changes in sound velocity that are measured by the transducers on the catheters. The change is then calculated by the Transonic software, giving the result of Qa (ml/min). If Qa was unable to be obtained by the method, it was measured by the variable pump flow-based Doppler ultrasound method (Am J Kidney Dis 21:457-471, 1993). CO was measured by injecting 30 ml saline (37° C.) into the venous catheter without reversing the tubing lines. TPR was calculated by computer software by dividing the mean arterial blood pressure by CO.

Statistical Analyses

Data management and statistical analysis were done using the SPSS statistical software (version 11.0; USA). Distributions of continuous variables in groups were expressed as mean±SD and compared by Student's t-test. All data have been tested for normal distribution before using t-tests. Categorical variables, such as the frequency of AV fistula malfunction in the treatment group and controls were analyzed by the chi-square test. The 95% confidence interval for every variable was also calculated. Survival curves of unassisted patency of AV fistula were calculated by the Kaplan-Meier method and compared by the log-rank test. A statistically significant value was P less than 0.05.

Patient Characteristics

145 patients were enrolled in the study. Among them, 73 patients were randomly distributed to the control group and 72 patients to the group treated by FIR. As listed in Table 3, there was no difference in the demographic and clinical characteristics between the two groups of HD patients. During follow-up, 1 patient receiving FIR therapy and 4 patients in the control group underwent creation of another vascular access because of the poor response to angioplasty. In addition, patients were censored at the time of renal transplantation (n=3), death with a functioning access (n=5), shifting to peritoneal dialysis (n=4), or loss of follow-up (n=1). This study was terminated on Dec. 31, 2005. Finally, 127 patients completed the study with 64 patients in the control group and 63 patients in the group treated with FIR therapy.

TABLE 3

| | Control group | FIR group | P value |
|---|---|---|---|
| N | 73 | 72 | |
| Age (years) | 59.2 ± 15.0 | 61.9 ± 14.4 | 0.87 |
| Gender (male) | 38 (52.1%) | 37 (51.4%) | 0.94 |
| HD duration (months) | 79.2 ± 42.2 | 85.2 ± 41.1 | 0.76 |
| Prevalence of hypertension | 39 (53.4%) | 40 (55.6%) | 0.80 |
| Prevalence of diabetes mellitus | 24 (32.9%) | 25 (34.7%) | 0.81 |
| History of AV fistula | 34 (46.6%) | 33 (45.8%) | 0.81 |
| malfunctiona | | | |
| Number of angioplasty | 20/46 | 20/49 | 0.87 |
| (patients/procedures) | | | |
| Number of surgical revision | 14/19 | 13/20 | 0.56 |
| (patients/procedures) | | | |
| Duration of AV fistula (months) | 58.7 ± 33.8 | 56.8 ± 36.4 | 0.91 |
| Creation of another AV | 4 (5.5%) | 1 (1.4%) | 0.18 |
| fistula (n)^{b} | | | |
| Renal transplantation (n) | 1 (1.4%) | 2 (2.8%) | 0.55 |
| Peritoneal dialysis (n) | 2 (2.7%) | 2 (2.8%) | 0.99 |
| Death with functioning AV | 2 (2.7%) | 3 (4.2%) | 0.64 |
| fistula (n) | | | |
| Loss to follow-up (n) | 0 | 1 (1.4%) | 0.31 |

^{b}Creation of another AV fistula during this 1-year study

The Effect of Single Session of FIR Therapy on the Hemodynamic Parameters in HD Patients

There was no significant difference in most of the hemodynamic parameters (such as blood pressure, cardiac output, total peripheral resistance) between the single HD session with or without FIR treatment for the 72 HD patients. However, the incremental change of access flow [Δ(Qa2−Qa1)] of these patients in the single HD session with FIR therapy was significantly higher than that without FIR therapy (13.2±114.7 vs. −33.4±132.3 ml/min; P=0.021), the results are listed in Table 4.

TABLE 4

| | HD session | HD session | |
|---|---|---|---|
| | without FIR | with FIR | P value |
| SBP1 (mmHg) | 134.2 ± 23.9 | 130.7 ± 19.0 | 0.86 |
| SBP2 (mmHg) | 132.1 ± 19.1 | 129.4 ± 17.3 | 0.74 |
| Δ(SBP2 − SBP1) (mmHg) | −2.1 ± 14.5 | −1.3 ± 14.8 | 0.68 |
| Qa1 (ml/min) | 976.6 ± 491.2 | 967.8 ± 421.0 | 0.94 |
| Qa2 (ml/min) | 943.2 ± 472.2 | 981.0 ± 430.8 | 0.36 |
| Δ(Qa2 − Qa1) (ml/min) | −33.4 ± 132.3 | 13.2 ± 114.7 | 0.021 |
| CO1 (L/min) | 4.75 ± 1.38 | 4.72 ± 1.10 | 0.91 |
| CO2 (L/min) | 4.45 ± 1.25 | 4.34 ± 1.16 | 0.88 |
| Δ(CO2 − CO1) (L/min) | −0.30 ± 0.75 | −0.38 ± 0.74 | 0.77 |
| Qa1/CO1 | 0.223 ± 0.090 | 0.217 ± 0.076 | 0.78 |
| Qa2/CO2 | 0.216 ± 0.082 | 0.235 ± 0.099 | 0.42 |
| Δ[(Qa2/CO2) − | −0.007 ± 0.057 | 0.018 ± 0.056 | 0.027 |
| (Qa1/CO1)] | | | |
| TPR1 (mmHg × min/L) | 21.29 ± 5.56 | 20.86 ± 4.81 | 0.89 |
| TPR2 (mmHg × min/L) | 22.86 ± 6.09 | 22.67 ± 5.57 | 0.92 |
| Δ(TPR2 − TPR1) | 1.56 ± 4.35 | 1.82 ± 3.98 | 0.81 |
| (mmHg × min/L) | | | |

SBP: systolic blood pressure;
Qa: access flow;
CO: cardiac output;
TPR: total peripheral resistance;
1 indicates the timing of measuring the parameter is within the first hour after initiation of HD session or immediately before FIR treatment;
2 represents the timing of measuring the parameter is 40 minutes after timing 1 or immediately after FIR treatment; NS: not significant

The Effect of One Year of FIR Therapy on Access Flow and Unassisted Patency of AV Fistula in HD Patients

In comparison with the control group, the HD patients receiving FIR treatment thrice a week for a year had higher incremental values of the following changes of access flow regarding both the initial and final HD sessions, including (1) Δ(Qa4−Qa3) [36.2±82.4 vs. −12.7±153.6 ml/min; P=0.027], (2) Δ(Qa3−Qa1) [36.3±166.2 vs. −51.7±283.1 ml/min; P=0.035], (3) Δ(Qa4−Qa2) [99.2±144.4 vs. −47.5±244.5 ml/min; P<0.001], and (4) Δ(Qa4−Qa2)−Δ(Qa3−Qa1) (ml/min) [62.9±111.6 vs. 4.1±184.5 ml/min; P=0.032], the results are listed in Table 5. As listed in Table 4, the FIR group had a lower incidence of AV fistula malfunction [12.5% (9/72) vs. 30.1% (22/73); P<0.01]. Some patients experienced multiple episodes of AV fistula malfunction during this study. Thus, relative incidences of AV fistula malfunction (number of incidences per patient months of follow-up) were calculated. The relative incidence of AV fistula malfunction in the FIR group (1 episode per 67.7 patient-months) was significantly lower than that in the control group (1 episode per 26.7 patient-months; P=0.03; Table 4).

TABLE 5

| | Control group | FIR group | P value |
|---|---|---|---|
| Case number completing study | 64 | 63 | — |
| Qa1 (ml/min) | 992.8 ± 473.8 | 975.2 ± 421.9 | 0.58 |
| Qa2 (ml/min) | 975.9 ± 444.3 | 948.6 ± 432.7 | 0.26 |
| Δ(Qa2 − Qa1) (ml/min) | −16.9 ± 130.3 | −26.6 ± 105.5 | 0.72 |
| Qa1/CO1 | 0.206 ± 0.088 | 0.204 ± 0.078 | 0.90 |
| Qa2/CO2 | 0.223 ± 0.095 | 0.225 ± 0.107 | 0.93 |
| Δ[(Qa2/CO2) − (Qa1/CO1)] | 0.017 ± 0.060 | 0.021 ± 0.059 | 0.75 |
| Qa3 (ml/min) | 941.1 ± 367.9 | 1011.6 ± 447.0 | 0.14 |
| Qa4 (ml/min) | 928.4 ± 387.5 | 1047.8 ± 463.2 | 0.07 |
| Qa3/CO3 | 0.194 ± 0.071 | 0.213 ± 0.077 | 0.16 |
| Qa4/CO4 | 0.197 ± 0.074 | 0.237 ± 0.079 | 0.004 |
| Δ(Qa4 − Qa3) (ml/min) | −12.7 ± 153.6 | 36.2 ± 82.4 | 0.027 |
| Δ[(Qa4/CO4) − (Qa3/CO3)] | 0.003 ± 0.047 | 0.024 ± 0.033 | 0.004 |
| Δ(Qa3 − Qa1) (ml/min) | −51.7 ± 283.1 | 36.3 ± 166.2 | 0.035 |
| Δ[(Qa3/CO3) − (Qa1/CO1)] | −0.012 ± 0.064 | 0.009 ± 0.040 | 0.031 |
| Δ(Qa4 − Qa2) (ml/min) | −47.5 ± 244.5 | 99.2 ± 144.4 | <0.001 |
| Δ[(Qa4/CO4) − (Qa2/CO2)] | −0.026 ± 0.056 | 0.013 ± 0.060 | <0.001 |
| Δ(Qa4 − Qa2) − Δ(Qa3 − Qa1) (ml/min) | 4.1 ± 184.5 | 62.9 ± 111.6 | 0.032 |
| Δ[(Qa4/CO4) − (Qa2/CO2)] − Δ[(Qa3/CO3) − | −0.014 ± 0.068 | 0.004 ± 0.068 | 0.049 |
| (Qa1/CO1)] | | | |
| Case number starting study | 73 | 72 | — |
| Patients with new AV fistula malfunction | 22 (30.1%) | 9 (12.5%) | <0.01 |
| (%) | | | |
| Patients with thrombosis of AV fistula | 6 (8.2%) | 2 (2.8%) | 0.15 |
| Patients with intervention of AV fistula | 16 (21.9%) | 7 (9.7%) | 0.044 |
| Total observations (patient-months) | 802.4 | 812.6 | — |
| New episodes of AV fistula malfunction | 30 | 12 | — |
| during study | | | |
| Episode of AV fistula malfunction/patient- | 1/26.7 | 1/67.7 | 0.03 |
| months | | | |

Qa1 indicates the access flow measured within first hour after the initiation of the HD session immediately before the commencement of this study.
Qa2 indicates the access flow measured 40 minutes after Qa1 measurement during the HD session immediately before the commencement of this study.
Qa3 indicates the access flow measured before FIR treatment or within first hour after the initiation of the HD session when the study was completed.
Qa4 indicates the access flow measured 40 minutes after Qa3 or immediately after FIR treatment during the HD session when the study was completed.
Δ(Qa4 − Qa3) represents the sum of the thermal effect by FIR and the hemodynamic effect by HD on the change of Qa.
Δ(Qa3 − Qa1) represents the non-thermal effect of one year of FIR on the change of Qa.
Δ(Qa4 − Qa2) represents the sum of the thermal effect (for 40 minutes) and the non-thermal effect (for one year) by FIR on Qa.
[Δ(Qa4 − Qa2) − Δ(Qa3 − Qa1)] represents the thermal effect of 40 minutes of FIR on the change of Qa.

Access Survival

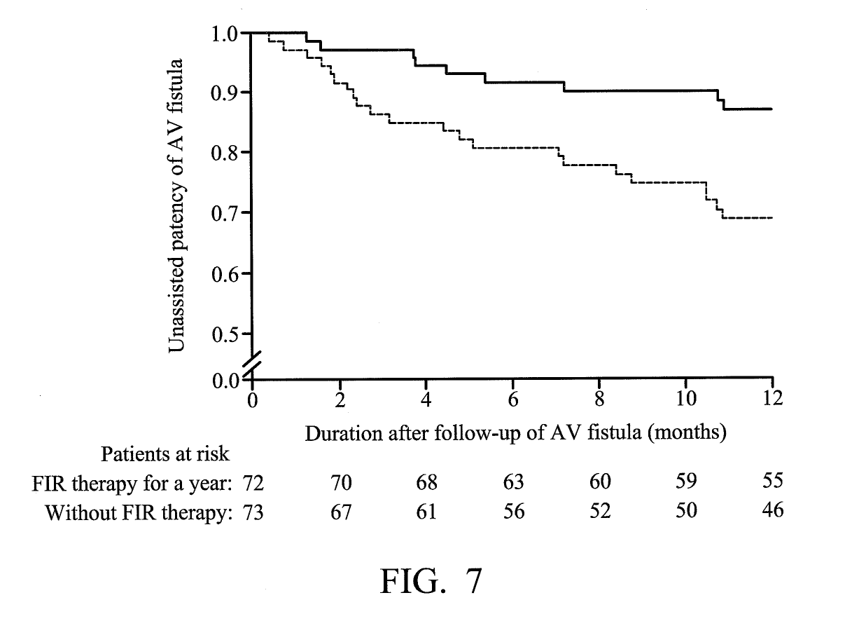

Eight patients of the FIR group and five patients of the control group were censored because of reasons other than AV fistula malfunction, such as renal transplantation, death with a functioning access, switching to peritoneal dialysis, and loss to follow-up. Consequently, the expected patient numbers used as the denominator for calculation of the unassisted patency of AV fistula was adjusted to 64 for the FIR group and 63 for the control group. Referring to FIG. 7, the unassisted patency of AV fistula at one year was significantly better in the FIR group than in the control group [85.9% (55 of 64) versus 67.6% (46 of 68); P<0.01 by Log rank test]. Concerning the safety issue of FIR therapy, no patient complained of any side effect, such as skin burn or allergy to the FIR therapy throughout the entire course of the study.

### Example 8

### Far Infrared Therapy Improves Access Flow and Endothelial Function of Newly-Created Arteriovenous Access in Patients with End Stage Renal Disease

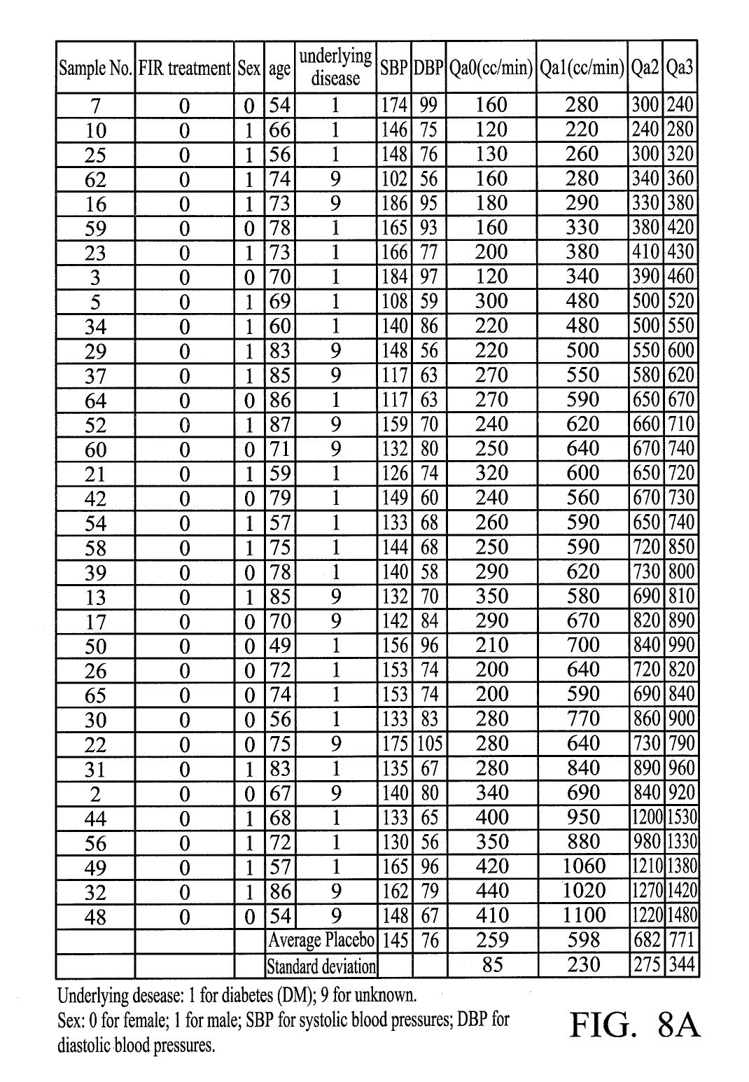

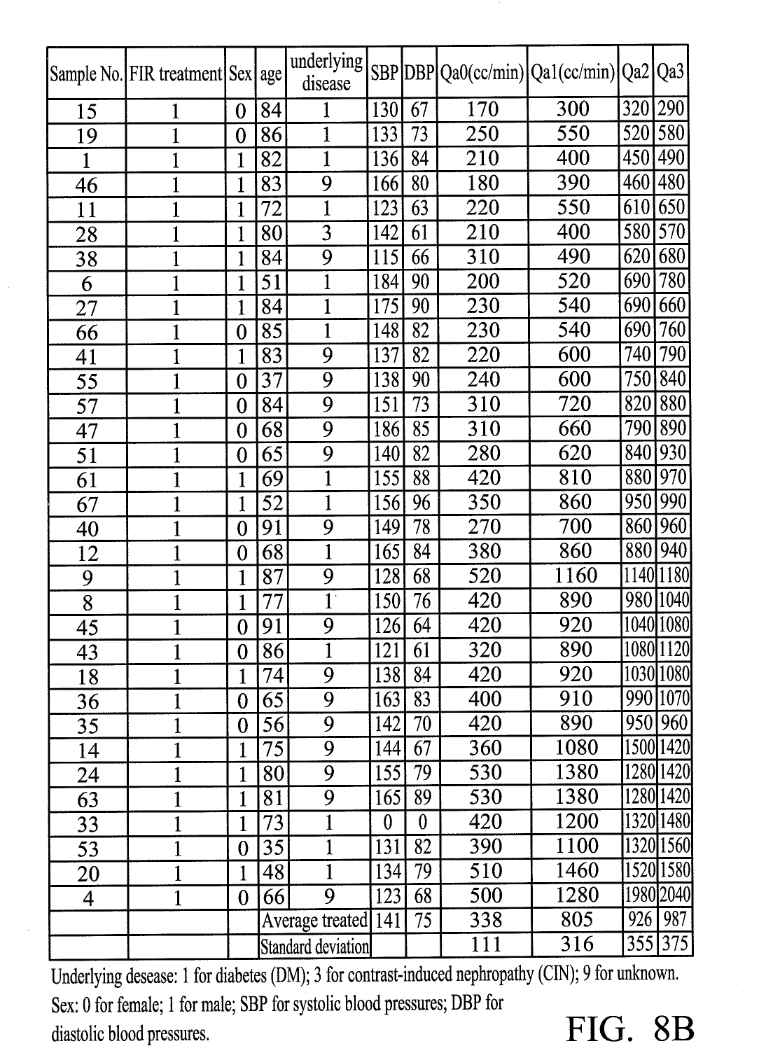

75 patients (in chronic kidney disease (CKD) stage 5) were randomly allocated to treatment group (receiving FIR therapy for 40 minutes three times weekly for 3 months, N=37) and control group (without FIR therapy, N=38). Access flow of arteriovenous fistula was measured by Doppler ultrasonography at 4 timings, including 2 days, 1 month, 2 and 3 months after vascular surgery. Markers of Endothelial function, including asymmetric dimethyl arginine (ADMA) and L-arginine (Wu, C C., et al., Plasma ADMA predicts restenosis of arteriovenous fistula, J Am Soc Nephrol 20:213-222, 2009; and Kamada Y., et al., Vascular endothelial dysfunction resulting from L-arginine deficiency in a patient with lysinuric protein intolerance, 1. Clin. Invest. 108: p. 717-724 (2001)), were measured both immediately before and 3 months after the creation of arteriovenous fistula. At the end, 67 patients completed the study, including 33 in FIR group and 34 controls. The result of raw data was shown in FIG. 8A˜8B.

Blood flow more than 500 ml/min was regarded as the maturation of AV fistula. Table 6 showed the ratio of the samples with blood flow more than 500 ml/min to all samples based on the raw data in FIG. 8A˜8B. The failure to AV fistula maturation accordingly was 50%.

TABLE 6

| | Control group | FIR group |
|---|---|---|
| Case numbers completing study | 34 | 33 |
| Qa1 (ml/min) | 70.5% | 84.4% |
| | (24/34) | (28/33) |
| Qa2 (ml/min) | 76.4% | 90.9% |
| | (26/34) | (30/33) |
| Qa3 (ml/min) | 76.4% | 90.9% |
| | (26/34) | (30/33) |

In addition, FIR group had lower values of incremental change of ADMA as well as higher values of Qa at all of the 4 timings and incremental change of L-arginine, thus leading to a higher incremental change in the ratio of L-arginine to ADMA 3 months later (as shown in table 7). In conclusion, 3 months of FIR therapy improves endothelial function and access flow of newly created AV fistula in patients with CKD stage 5.

TABLE 7

| | Control group | FIR group | P value |
|---|---|---|---|
| Case numbers completing | 34 | 33 | — |
| study | | | |
| Qa0 (ml/min) | 259.1 ± 85.5 | 337.9 ± 111.3 | 0.002 |
| Qa1 (ml/min) | 597.9 ± 230.1 | 805.2 ± 315.7 | 0.003 |
| Qa2 (ml/min) | 681.8 ± 275.2 | 925.8 ± 354.6 | 0.002 |
| Qa3 (ml/min) | 770.6 ± 344.0 | 987.3 ± 375.1 | 0.016 |
| ΔL-arginine 3-0 (μM) | −0.7 ± 2.9 | 2.2 ± 5.2 | 0.007 |
| ΔADMA 3-0 (μM) | 0.01 ± 0.05 | −0.05 ± 0.07 | <0.001 |
| (L-arginine3/ADMA3)/(L- | 0.98 ± 0.08 | 1.09 ± 0.12 | <0.001 |
| arginine0/ADMA0) | | | |

Qa0/Qa1 or Qa2 or Qa3 indicate the access flow measured within 2 days/one or two or three months after the creation of AV fistula;
ADMA0 or L-arginine0 indicates the concentration measured before creation of AV fistula;
ADMA3 or L-arginine3 indicates the concentration measured 3 months after creation of AV fistula.

### Example 9

### The Effect of FIR Therapy on Peripheral Artery Diseases in Maintenance Hemodialysis Patients

155 hemodialysis (HD) patients were enrolled to receive ankle-brachial index (ABI) and brachial-ankle pulse wave velocity (baPWV), the markers for evaluating the risk of peripheral artery diseases (PAD) and atherosclerosis. Herein, 35 patients (22.6%) who had abnormal ABI (<0.9) were allocated to FIR treatment. For these 35 patients with abnormal ABI, 40 minutes of single treatment of FIR during HD sessions was associated with higher post-dialytic values and intra-dialytic changes of ABI than those in HD sessions without receiving 40 minutes of FIR treatment. The results were shown in Table 8 and had demonstrated that the FIR therapy can improve access flow and unassisted patency of AV fistula

TABLE 8

| | HD session | HD session with | |
|---|---|---|---|
| Indication | without FIR | FIR | P value |
| ABI-BD | 0.71 ± 0.09 | 0.72 ± 0.08 | 0.94 |
| ABI-AD | 0.65 ± 0.10 | 0.77 ± 0.11 | 0.03 |
| ABI (AD − BD) | −0.06 ± 0.05 | 0.05 ± 0.06 | 0.02 |
| PWV-BD (cm/sec) | 1798 ± 214 | 1775 ± 209 | 0.86 |
| PWV-AD (cm/sec) | 1839 ± 232 | 1728 ± 197 | 0.08 |
| PWV(AD − BD) (cm/sec) | 41 ± 49 | −47 ± 42 | 0.06 |

ABI, ankle brachial index;
PWV, pulse wave velocity;
BD, before hemodialysis;
AD, after hemodialysis.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for improving patency of an arteriovenous fistula in a subject in need thereof, comprising irradiating the subject skin surface above the arteriovenous fistula with an electromagnetic wave from an emitter, wherein the electromagnetic wave has a wavelength of about 1.5 to 100 μm.

2. The method as claimed in claim 1, wherein the electromagnetic wave has a peak in a wavelength of about 5 to 8 μm.

3. The method as claimed in claim 1, wherein the irradiation is performed more than 10 minutes once time.

4. The method as claimed in claim 1, wherein the irradiation is performed at least once every two days.

5. The method as claimed in claim 1, wherein a power density of the emitter is lower than 1.3 W/cm².

6. The method as claimed in claim 1, wherein the distance between the emitter and the subject skin surface is about 0.1 to 60 cm.

7. The method as claimed in claim 1, wherein the subject skin surface after the irradiation reaches up to 40° C.

8. The method as claimed in claim 1, wherein the subject comprises a human or non-human mammal.

9. A method for preventing and/or ameliorating a peripheral artery disease in a subject in need thereof, comprising irradiating the subject skin surface above the site that the peripheral artery disease occurs with an electromagnetic wave from an emitter, wherein the electromagnetic wave has a wavelength of about 1.5 to 100 μm.

10. The method as claimed in claim 9, wherein the electromagnetic wave has a peak in a wavelength of about 5 to 8 μm.

11. The method as claimed in claim 9, wherein the irradiation is performed more than 10 minutes once time.

12. The method as claimed in claim 9, wherein the frequency of the irradiation is at least once every two days.

13. The method as claimed in claim 9, wherein a power density of the emitter is lower than 1.3 W/cm².

14. The method as claimed in claim 9, wherein the distance between the emitter and the subject skin surface is about 0.1 to 60 cm.

15. The method as claimed in claim 9, wherein the subject skin surface after the irradiation reaches up to 40° C.

16. The method as claimed in claim 9, wherein the subject comprises a human or non-human mammal.

17. A method for decreasing a rate of failure of arteriovenous fistula maturation in a subject in need thereof, comprising irradiating the subject skin surface above the arteriovenous fistula with an electromagnetic wave from an emitter, wherein the electromagnetic wave has a wavelength range of about 1.5 to 100 μm.

18. The method as claimed in claim 17, wherein the electromagnetic wave has a peak in a wavelength of about 5 to 8 μm.

19. The method as claimed in claim 17, wherein the irradiation is performed more than 10 minutes once time.

20. The method as claimed in claim 17, wherein the irradiation is performed at least once every two days.

21. The method as claimed in claim 17, wherein a power density of the emitter is lower than 1.3 W/cm².

22. The method as claimed in claim 17, wherein the distance between the emitter and the subject skin surface is about 0.1 to 60 cm.

23. The method as claimed in claim 17, wherein the subject skin surface after the irradiation reaches up to 40° C.

24. The method as claimed in claim 17, wherein the subject comprises a human or non-human mammal.
